(19) United States
(12) Patent Application Publication
(10) Pub. No.: US 2009/0198262 A1
(43) Pub. Date: Aug. 6, 2009

(54) DISPOSABLE TONGUE SCRAPER

(75) Inventors: Kenneth Rosenblood, Los Angeles, CA (US); Christopher Quan, North Hills, CA (US); Scott Mejia, Los Angeles, CA (US); Brendan McLoughlin, Los Angeles, CA (US)

Correspondence Address: DISCUS DENTAL, LLC, 8550 HIGUERA STREET, CULVER CITY, CA, 90232, US

(73) Assignee: Discus Dental, LLC, Culver City, CA (US)

(21) Appl. No.: 11/963,510

(22) Filed: Dec. 21, 2007

Related U.S. Application Data
(60) Provisional application No. 60/877,345, filed on Dec. 26, 2006.
(60) Provisional application No. 60/877,337, filed on Dec. 26, 2006.

(51) Int. Cl. A61B 17/24

(52) U.S. Cl. 606/161

(57) ABSTRACT

The present invention relates to a dental implement such as tongue scraper that is disposable and is adapted for prophylactic and/or therapeutic action for the oral cavity. The dental implement may include a tongue scraper, a dental floss, a toothpick, a toothbrush or combinations thereof. The dental implement may include a strip having a longitudinal dimension that is longer than its traverse dimension. The strip may be coated with a coating of a mixture including at least one medicament and a water soluble or erodible carrier. In one embodiment, the coating may be present on at least one of the longitudinal edges, for example, of a tongue scraper, toothpick or a dental floss. In another embodiment, the coating may be present on at least one of the traverse edges, for example, of a tooth pick or the bristles of a toothbrush. In a further embodiment, the coating may be present on a combination of edges. The integrity of the implement, such as a scraper and/or its edge or edges may be reinforced by the coating of substance or agent for sufficient integrity, and the scraper or its edge and/or the coating, for example, are adapted to degrade or break down as the substance or agent are released during use and/or upon contact with water and/or saliva. The dental implement may be exposed for use, and is appropriately stored prior to use.

### FIELD OF THE INVENTION

This invention relates broadly to improvements in the field of oral hygiene. Specifically, this invention relates to a dental apparatus for use in oral hygiene.

### BACKGROUND OF THE INVENTION

The human oral cavity is susceptible to many conditions which may adversely affect oral health, appearance and/or perception, such as dental carries, halitosis and/or gum disease.

The human tongue, for example, is not a smooth surface and has numerous protuberances, e.g., papillae, which convey to the brain the senses of taste and touch. However, due to the non-uniform surface of the tongue, it may also be a breeding ground for microorganisms such as bacteria and may retain food debris, volatile sulfur compounds (which may be a major cause of halitosis) and dead cells. Over time, some of the collected material becomes a soft plaque which may be another cause of bad breath and is also known to attack the teeth and gums. Tests have shown that daily scraping to reduce the amount of coating on the tongue may eliminate much of the undesirable microbes and sulfur compounds, and thus may significantly inhibit plaque formation on the teeth in the long term, and substantially reduce halitosis in the short term.

Various dental instruments exist for prophylactic and/or therapeutic action on the oral cavity. These instruments include toothbrushes, toothpicks, dental floss, tongue scrapers and the like. A wide variety of tongue scrapers, for example, are known in the dental hygiene arts. Some tongue scrapers incorporate rigid blade-like structures while others may be flexible, or have brush-like structures. The prior art also includes tongue scrapers that are both reusable and disposable. Tongue scrapers may also include flavorings or fragrances.

While these devices may fulfill their respective objectives, there still exists a need for a dental implement, for example, a tongue scraper, which may effectively deliver therapeutics to the oral cavity during the course of its use, remove plaque coating, reduce bacteria growth, reduce halitosis and lead to better overall mouth cleaning and oral hygiene that is also simple and disposable.

### SUMMARY OF THE INVENTION

The present invention relates to a dental implement that is disposable and is adapted for prophylactic and/or therapeutic action for the oral cavity. The dental implement may include a tongue scraper, a dental floss, a toothpick, a toothbrush, or combinations thereof.

The dental implement may include a strip having a longitudinal dimension that is longer than its traverse dimension. The strip may be coated with a coating of a mixture including at least one medicament and a water soluble or erodible carrier. In one embodiment, the coating may be present on at least one of the longitudinal edges, for example, of a tongue scraper, toothpick or a dental floss. In another embodiment, the coating may be present on at least one of the traverse edges, for example, of a tooth pick or the bristles of a toothbrush. In a further embodiment, the coating may be present on a combination of edges.

The strip(s) of the dental implement may be, in general, composed of any material that may be suitable for the primary purpose of the implement. The strip(s) may further be adapted to retain a coating. The surface of the strip(s) may, for example, be roughened, pitted or otherwise have a modified surface that may better retain a coating.

In one aspect, the coating may be a formed on the strip(s) of a dental implement by bringing a dissolved form of the coating into contact with the appropriate surface(s) and drying the solution to form the coating. In one embodiment, the coating may be substantially dry. In other embodiments, variable amounts of solvent may be retained in the coating, resulting in different characteristics of the coating. In general, the coating is substantially in the dry state to an extent that it may remain on the surface(s) of the dental implement without running, dripping and/or otherwise falling off the surface(s). The coating of the implement may generally occur during manufacture and the implement may be packaged and ready for use.

In one exemplary embodiment of the invention, a dental implement may include a tongue scraper made of a generally planar strip of a material having at least one edge. The edge may be coated or impregnated with a substance or agent that is releasable upon contact with water and/or saliva. The integrity of the scraper and/or its edge or edges may be reinforced by the coating or impregnation of substance or agent for sufficient integrity, and the scraper or its edge and/or the coating, for example, are adapted to degrade, break down or dissolve as the substance or agent is released during use and/or upon contact with water and/or saliva. In one aspect, the substance or agent, may be a film forming material that is dissolvable or erodible upon contact either with water and/or saliva, or a substance or agent that is not itself dissolvable or erodible upon contact either with water and/or saliva and may be combined with a film forming material for coating onto the tongue scraper.

As noted above, the coating may be adapted to provide substances or agents for delivery to various portions of the oral cavity.

A substance or agent may generally include therapeutic agents, such as those adapted for freshening breath, reducing bad breath by killing bacteria that are associated with bad breath, interfering with microbial viability in the presence or absence of light as their principle mode of action, or simply reducing gag reflex. The coating may also include flavoring agents to improve the taste of the therapeutic agents, carriers, binders, adhesives, tackifying agents, and similar. The coating may be completely soluble, or the coating may be erodible upon contact with water and/or saliva.

In exemplary aspects, the coating may be a composition that includes a water soluble or water dispersible carrier in which a medicament may be dispersed. The water soluble or dispersible carrier may be, for example, a soluble polymeric material. Such materials include, but are not limited to, hydrophilic materials including polysaccharides such as carrageenan, chondroitin sulfate, glucosamine, pullulan, soluble cellulose derivatives such as hydroxypropyl cellulose and hydroxymethyl cellulose, polyacrylic acid, polyvinyl alcohol, polyethylene glycol (PEG), polyethylene oxide (PEO), ethylene oxide-propylene oxide co-polymer, polyvinylpyrrolidone (PVP) and/or any other appropriate material.

In an exemplary embodiment, the water soluble carrier included is pullulan. Pullulan is a soluble polysaccharide polymer derived from a fungal organism composed of triose sugar units. Pullulan coatings may be a self adherent, flexible and/or relatively high strength film that may resiliently remain on the applied surface(s) and that dissolves very quickly on contact with a wet or moist environment. They are also useful in encapsulating other materials, as pullulan has oxygen barrier properties and is thus further useful for preserving the encapsulated material(s).

In general, a solution of pullulan may be made in water and medicaments and additives may be mixed into the solution to disperse them. The solution may then be applied to the surface(s) of the strip(s) of a dental implement, which upon drying may form an adherent film with encapsulated medicament. The coating may then dissolve on contact with a wet or moist environment such as the oral cavity during use and release the encapsulated medicament and/or other additives.

In some embodiments, it may be desirable for the coating to have a slower rate of dissolution such that the release of medicament and/or other additives may be prolonged. It may further be desirable for only a portion of the coating to dissolve at any one time such that the dental implement may be used more than once with the action of the coating. The coating may, for example, incorporate additives that may serve to slow the rate of dissolution of the water soluble carrier.

In one embodiment, pullulan may be mixed with a relatively insoluble additive to form a coating that may be erodible, or slowly dissolving, if desired. The degree of solubility or erodibility depends on the ratio of pullulan and the insoluble additive. The combination may be, for example, pullulan and high molecular weight PEG or PEO.

According to one embodiment, the coating or impregnation may be introduced onto the surface(s) during the manufacturing process. In one aspect, the substance or agent may be incorporated into the base material of the dental implement, for example, the tongue scraper(s). In another aspect, the substance or agent may be coated onto the base material after the base material has been formed, by, for example, chemical attachment or physical attachment. In yet another aspect, the substance or agent may be present as a coating which is laminated to the base material during the manufacturing of the dental implement, for example tongue scraper.

In one embodiment, the dental implement is designed to have sufficient integrity for one-use, thus further improving oral hygiene, as reusable, for example, scrapers may tend to be difficult and/or expensive to sterilize to minimize or eliminate, or may also tend to promote microbial growth after rinsing and/or improper drying.

In another embodiment, the dental implement such as a tongue scraper may have sufficient integrity to be used for a longer period, for example, about a week. The tongue scraper may include edges adapted for cleaning the surface of the tongue and the edges may be made of a coating material of sufficient hardness, and at the same time flexible enough, for the edges to be used to scrape the tongue. The edge material may be adapted to erode and/or dissolve. As these edges erode and/or dissolve over a period of time's worth of use, the user may simply disposed of the tongue scraper. The edge material may be further adapted to retain and release a substance or agent in a manner similar to the coating or impregnation discussed above.

In yet another embodiment, the entire edge of the dental implement such as the tongue scraper may be coated with an erodible coating that may be eroded during use. The coating may be colored so that the user could visually tell when the coating is gone and that the tongue scraper needs to be replaced.

The present invention further relates to a dental implement having a tongue scraper which is exposed for use, and is appropriately stored prior to use.

In one exemplary embodiment of the invention, a dental implement may include a housing having a distal end, and a proximal end. The housing may have a substantially hollow interior. In one embodiment, the tongue scrapers may be present in a roll inside the substantially hollow housing during storage. The roll may be of a sufficient length when extended to form at least one tongue scraper. The housing may include an opening at one end or along one side. When the opening is at the end, the tongue scraper may be pulled out as an extension of the housing, or perpendicular to the housing. After use, it may be torn off and disposed of to reduce cross-contamination. When the opening is along one side, the tongue scraper may be pulled out in a perpendicular direction. The opening may further be fitted with a door. The roll may be fixedly or slidably attached to the proximal end of the housing, anywhere inside the housing, or to the distal end of the housing.

In one exemplary embodiment of the invention, a substance or agent may be stored separately from the dental implement, such as, tongue scraper(s) and introduced just prior to use. In this embodiment, the scraper and/or its edge or edges may have sufficient integrity for at least one use in the absence of the therapeutic agent, but may gradually lose its integrity during use, or upon contact with water and/or saliva.

In one aspect, a substance may be stored as a liquid or gel and coated onto a dental implement, such as a tongue scraper prior to use by means of an applicator. In another aspect, a substance may be stored as a solid and deposited onto the tongue scraper(s) prior to use. According to one embodiment, an applicator may be manually operated. According to another embodiment, an applicator may be automatic. According to one aspect, the applicator may be a separate device. According to another aspect, it may be included with the dental implement such as a tongue scraper assembly. According to a further aspect, it may be part of a dispensing device.

In one embodiment, the surface of, for example, a tongue scraper, may be adapted to at least partially retain a substance or agent when it is deposited on it prior to use such that the substance or agent may be readily and efficiently conveyed to the oral cavity without spilling, dripping or otherwise escaping the tongue scraper(s) prior to use. In another embodiment, the tongue scraper(s) may include a modified surface that may be adapted to better retain a substance. In yet another embodiment, the surface of at least an edge of the tongue scraper(s) may be porous and/or otherwise absorptive such that it may better retain a substance.

In one aspect, the tongue scraper may be flexible such that it may be bent and/or otherwise deformed for more effective use, storage, and/or dispensing. In one embodiment, the tongue scraper may be present in a roll. The roll may be of a sufficient length when extended to form at least one tongue scraper. In an exemplary embodiment, it may be torn off and disposed of to reduce cross-contamination. The roll may feature perforations, reduced span and/or any other appropriate features between individual tongue scrapers such that they may be easily separated for use while still remaining a continuous roll during storage and dispensing.

The present invention further relates to a dental implement including a dispensing device adapted to store and dispense, fro example, tongue scraping inserts for use with a handle or set of handles. In one exemplary aspect, the tongue scraper may be a compound device.

In one embodiment, the tongue scraper may include at least one component that may be utilized to articulate and/or handle the tongue scraper as a whole and at least one other component that may be utilized on the oral cavity.

The tongue scraper may include, for example, a set of handles that may be adapted to retain at least one tongue scraping insert. The tongue scraping insert may be substantially similar to the tongue scrapers discussed above and below, but may be adapted to utilize articulating and/or handling features of the set of handles rather than incorporating them into the insert. The handles may be reusable such that the insert may be replaced after each use. The handles may also incorporate features that may serve to bolster and/or support the tongue scraping insert. These features may include, for example, a flexible section that may be adapted to reinforce the tongue scraping insert.

In another embodiment, tongue scrapers or other dental implements may be present in a stack. The individual tongue scrapers may be attached as previously discussed. The length of tongue scrapers may be folded opposingly at each interface between individual tongue scrapers, for example, fan-folded, such that the stack may be the length of one tongue scraper.

In some aspects, the tongue scraper(s) or other dental implements may be stored and dispensed in individual packages. Packaging may include, but is not limited to, paper wrappers, plastic wrappers, foil wrappers, and/or any other appropriate packaging. It may be generally desirable for the packaging to be sealed such that the contents may be well isolated from the environment prior to use. It may also be generally desirable to utilize packaging that is not bulky and may occupy a very small volume during storage. The packaging may also incorporate features such that it may be easily opened by a user while still retaining its sealing properties during storage.

In other aspects the tongue scrapers may be stored and dispensed from a dispensing device. A dispensing device may, in general, store the tongue scrapers and dispense them in a manner that enables the user to easily isolate a single scraper for use.

In one embodiment, a dispensing device may be adapted to store and dispense dental implements such as tongue scrapers in a roll. In other embodiments, a dispensing device may be adapted to store and dispense tongue scrapers in a stack. In still other embodiments, a dispensing device may be provided that may store and dispense individually packaged tongue scrapers. In yet other embodiments, a dispensing device may be adapted to store and dispense tongue scraping inserts for use with a handle or set of handles.

In a further aspect, as also mentioned above, the housing may include features that may coat the tongue scraper as it is being pulled out of the housing with any substance as discussed above.

As noted above, it may be desirable to provide substances or agents on the tongue scraper for delivery and/or action on the oral cavity. The substance or agent, a carrier and/or any other additives or ingredients may be collectively referred to as a medicament.

The substance or agent may generally include therapeutic agents, such as those adapted for freshening breath, reducing bad breath by killing bacteria that are associated with bad breath, interfering with bacterial viability in the presence or absence of light as their principle mode of action, or simply reducing gag reflex. The medicament may also include flavoring agents to improve the taste of the therapeutic agents, carriers, binders, adhesives, tackifying agents, and similar.

In one exemplary embodiment, the tongue scraper may include formations to aid in its function of generally removing matter from the tongue surface and introducing therapeutics to the oral cavity. The tongue scraper may also include features that may enable it to be easily used, stored and/or disposed of.

In one aspect, the tongue scraper may include two end portions with a generally planar section disposed between the ends. The generally planar section may have at least one edge that may be adapted for use as a scraper. The scraping edge may include formations that may aid in its function, such as, for example, teeth, crenellations, ridges, and/or any other appropriate formations. Scrapers may be present on any of the edges.

In another aspect, the end portions may also be adapted to aid the user in grasping, handling and/or otherwise articulating the tongue scraper. The end portions may include, for example, handles, gripping surfaces, ring holders, handling bars, and/or any other appropriate formations.

In one embodiment, the tongue scraper may include at least one straight longitudinal edge. In another embodiment, the tongue scraper may include at least one serrated longitudinal edge. In a further embodiment, the tongue scraper may be a dual action tongue scraper including a comparatively hard scrape when the tongue scraper is bent one way, such that the tongue contacts a hard scraping surface which extends along one edge thereof; and a comparatively soft scrape when the tongue scraper is bent another way, such that the tongue contacts a soft scraping surface which extends along either the same or a different edge thereof. The hard scrape is generally more effective at removing the undesirable coating from the tongue, but the hard scrape may be too harsh for some people.

In one aspect, when the dual action tongue scraper is formed upon a common edge, the hard scraping surface and the soft scraping surface are separated by a generally flat portion of the common edge, so as to facilitate scraping of the tongue with only the desired one of the hard scraping surface and the soft scraping surface. In another aspect, one or both of the two edges may include a hard scraping surface and/or a soft scraping surface, if desired. In a further aspect, each of the two edges may either be configured as a smooth edge or a serrated edge, if desired. Further, in yet another aspect, each of the two edges may include either a substantially straight edge or a convex edge, if desired.

In general, the tongue scraper may be composed of any material amenable for a non-reusable scraper having sufficient integrity during use, and capable of retaining at least substance or agent prior to use. The scraper may include materials that are erodible, dissolvable, compostable, recyclable, and/or degradable. In one aspect, the material is a polymeric material. In exemplary embodiments, the tongue scraper may be composed of biopolymer material, which may include, but is not limited to, cellulose, polylactic acid (PLA), polyglycolic acid (PGA), and/or other biopolymeric material that may be appropriate. In general, it may be desirable to produce the tongue scraper from a material that may also be economically advantageous.

In one exemplary embodiment, the primary material for the tongue scraper may be a wood or plant fiber material such as, for example, paper, card stock, cardboard and/or any other appropriate wood or plant fiber material. The material may be present as a single layer in the tongue scraper, or it may be present as multiple layers or laminated.

In another embodiment, the primary material for the working surfaces of the tongue scraper may be a polypeptide or protein material, such as silk, collagen and/or other appropriate polypeptide or protein materials.

In a further embodiment, the material of the tongue scraper may be assembled in an intertwining fashion, such as being woven, spun, coiled, combination of a relatively insoluble material in a relatively soluble binder, and/or otherwise formed into a matrix-like configuration. The relatively insoluble material may be either the scraper forming material or the medicament.

In some embodiments, the tongue scraper may include a layer of medicament in a laminated structure.

As mentioned above, the medicament may include a film forming and/or soluble material for coating, impregnating and/or otherwise including the medicament with the tongue scraper. Suitable materials may include hydrophilic materials including polysaccharides such as carrageenan, chondroitin sulfate, glucosamine, pullulan, soluble cellulose derivatives such as hydroxypropyl cellulose and hydroxymethyl cellulose, polyacrylic acid, polyvinyl alcohol, polyethylene glycol (PEG), polyethylene oxide (PEO), ethylene oxide-propylene oxide co-polymer, polyvinylpyrrolidone (PVP), polycaprolactone, polyorthoesters, polyphosphazene, polyvinyl acetate, and polyisobutylene.

The medicament may be adapted to have a desirable rate of erosion and/or dissolution. The rate may be modified by the inclusion of hydrophobic and/or less soluble additives. Suitable materials may include, but are not limited to, those from the family of quaternary ammonium acrylate/methacrylate co-polymers, (Eudragit RS), cellulose and its lower solubility derivatives, such as butyl cellulose, hydroxybutyl cellulose and ethylhydroxyethyl cellulose, high molecular weight PEG or PEO or a combination thereof.

Similar materials and/or combinations may further be utilized to imbue the tongue scraper base material with erodible and/or dissolvable characteristics such that the tongue scraper may degrade during the course of its use. In embodiments where medicament may be included in the base material of the tongue scraper, such characteristics may further allow the medicament to be released in a controlled fashion.

Any of the discussion relating to tongue scrapers is equally applicable to other dental implements, where practicable.

Any of the above described apparatuses may also be fitted with a light source. The light source may also have therapeutic effects or may be adapted for enhancing therapeutic effects of the coatings.

Other variations and equivalent structures of the present invention are also contemplated to be within the scope of the present invention.

### BRIEF DESCRIPTION OF THE FIGURES

FIG. 1 is an embodiment of a dental implement of the present invention;

FIG. 1a illustrates alternative cross-sectional views of the dental implement of FIG. 1;

FIG. 2 is a partial cross-sectional view of the strip of a dental implement with a coating in one embodiment of the present invention;

FIG. 3 is a perspective view of a dental implement in the form of a coated tongue scraper in one embodiment of the present invention;

FIG. 3a is a perspective view of a dental implement in the form of a toothbrush with coated bristles in one embodiment of the present invention;

FIG. 3b is a perspective view of a dental implement in the form of a coated dental floss in one embodiment of the present invention;

FIG. 3c is a perspective view of a dental implement in the form of a coated toothpick in one embodiment of the present invention.

FIG. 4 is a top view of a tongue scraper in one embodiment of the present invention;

FIG. 5 is a perspective view of the tongue scraper of FIG. 1;

FIG. 5a illustrates a close up view of a tongue scraper with edges and serrations composed of a different material than the base material;

FIGS. 6, 6a, 6b and 6c illustrate embodiments of handle portions in the present invention;

FIG. 7 illustrates tongue scrapers present in a roll in one embodiment of the present invention;

FIG. 7a illustrates dividing regions between individual tongue scrapers in one embodiment of the present invention;

FIG. 8 illustrates a stack of tongue scrapers in one embodiment of the present invention;

FIG. 8a illustrates a zig-zag stack of tongue scrapers in one embodiment of the present invention;

FIG. 9 illustrates a tongue scraper with a substance applied to the surface in one embodiment of the present invention;

FIG. 9a illustrates a tongue scraper with a substance applied to the surface with a covering in one embodiment of the present invention;

FIG. 9b illustrates the removal of the covering from the tongue scraper of FIG. 6a;

FIG. 10 illustrates a dispensing device in one embodiment of the present invention;

FIG. 10a illustrates a dispensing device in another embodiment of the present invention;

FIG. 10b illustrates a substance application device in one embodiment of the present invention;

FIG. 10c illustrates a substance application device in another embodiment of the present invention;

FIG. 11 illustrates a tongue scraper device with a handle and a tongue scraping insert;

FIG. 11a-c illustrate a tongue scraper device with a set of handles and a tongue scraping insert.

### DETAILED DESCRIPTION OF THE INVENTION

The detailed description set forth below is intended as a description of the presently exemplified device provided in accordance with aspects of the present invention and is not intended to represent the only forms in which the present invention may be practiced or utilized. It is to be understood, however, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein may be used in the practice or testing of the invention, the exemplified methods, devices and materials are now described.

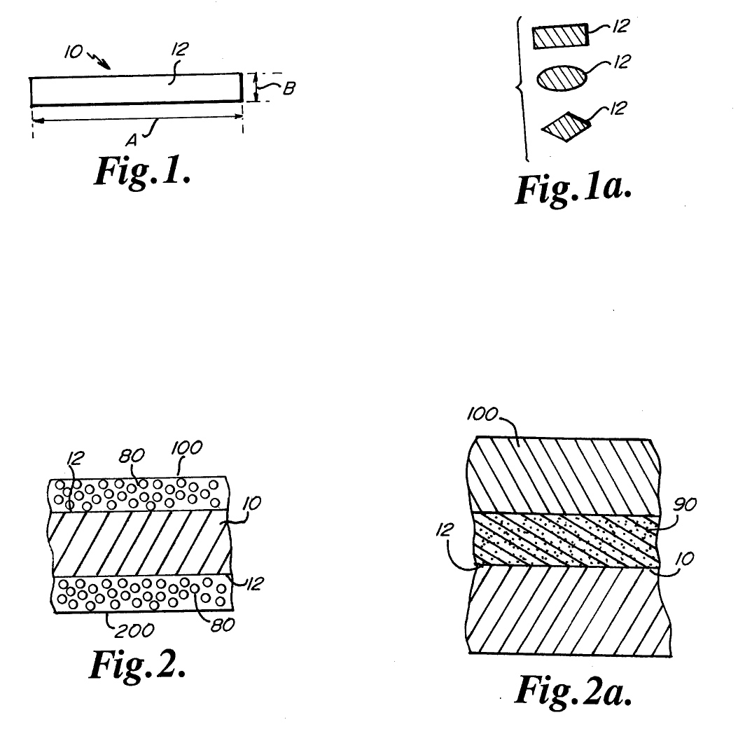

FIG. 1 illustrates an embodiment dental implement comprising strip 10. The strip 10 may be adapted to perform a dental prophylactic and/or therapeutic action in the oral cavity. The strip 10 may be of any size or proportion such that it may efficiently perform a desired action. In general, the strip 10 may have a longitudinal dimension A and a transverse dimension B, where the dimension A may be longer than the dimension B. The strip 10 may further have a surface 12 and it may also have a cross-section of any desirable shape, such as those illustrated in FIG. 1a. The cross-section may in general form edges and/or working portions along the longitudinal span of the strip 10.

In general, the strip 10 may be composed of generally disposable material. In one aspect, the material is a polymeric material. In exemplary embodiments, the strip 10 may be composed of biopolymer material. Biopolymers may be derived from an organism or may be produced synthetically. In general, biopolymers may be advantageous due to their biodegradation and biocompatibility properties. Suitable biopolymers may include those composed of biocompatible monomers, such as glucose, lactic acid, glycolic acid and/or other appropriate monomers. It may be advantageous to utilize such materials as their degradation generally may not produce any toxic or otherwise undesirable products. Exemplary materials for the strip 10 may include, but is not limited to, cellulose, polylactic acid (PLA), polyglycolic acid (PGA), and/or other biopolymeric material that may be appropriate. In general, it may be desired to produce the strip 10 from a material that may be economically advantageous, degradable and/or recyclable, particularly biodegradable, and biocompatible. The material may also be selected such that it may effectively and efficiently affect the desired prophylactic and/or therapeutic action.

In an exemplary embodiment of the invention, the surface 12 of the strip 10 has a coating thereon. FIG. 2 illustrates a partial cross-sectional view of an embodiment of a strip 10 with coatings 100 and 200 on the surface 12. The coatings 100 and 200 may be separate coatings on separate portions of the surface 12, or they may also form part of a single continuous coating. The coating 100, 200 may, for example, be present on some or all of the edges and/or working portions or on the entire surface 12 of the strip 10.

In one aspect, the coating 100, 200 may be a formed on the strip 10 by bringing a dissolved form of the coating 100, 200 into contact with the surface 12, for example, by contact coating, dip coating, curtain coating, extrusion coating, or similar application methods, and drying the solution to form the coating 100, 200. In one embodiment, the coating 100, 200 may be substantially dry. In other embodiments, variable amounts of solvent may be retained in the coating 100, 200, resulting in different characteristics of the coating 100, 200. In general, the coating 100, 200 is substantially in the dry state to an extent that it may remain on the surface 12 of the strip 10 without running, dripping and/or otherwise falling off the surface 12. The coating 100, 200 of the implement may generally occur during manufacture and the implement may be packaged and ready for use.

In general, the coating 100, 200 may be adapted to provide substances or agents for delivery to various portions of the oral cavity. A substance or agent 80 may be retained within the coating 100, 200, as illustrated in FIG. 2. The substance or agent 80 may, for example, be encapsulated as finite, discrete particles or the substance or agent 80 may also be dissolved or finely distributed.

Alternatively, a substance or agent may be present as a layer, as illustrated in FIG. 2a. A substance or agent layer 90 may be deposited on the surface 12 of the strip 10. The coating 100 may further be present on top of the substance or agent layer 90 and may act to seal, stabilize and/or retain the layer 90.

A substance or agent may generally include therapeutic agents, such as those adapted for freshening breath, reducing bad breath by killing bacteria that are associated with bad breath, interfering with microbial viability in the presence or absence of light as their principle mode of action, or simply reducing gag reflex. The coating may also include flavoring agents to improve the taste of the therapeutic agents, carriers, binders, adhesives, tackifying agents, and similar. The coating may be completely soluble, or the coating may be erodible upon contact with water and/or saliva.

In exemplary aspects, the coating may be a composition that includes a water soluble carrier in which a substance or agent may be dispersed. The water soluble carrier may be, for example, a soluble polymeric material. Such materials include, but are not limited to, hydrophilic materials including polysaccharides such as carrageenan, chondroitin sulfate, glucosamine, pullulan, soluble cellulose derivatives such as hydroxypropyl cellulose and hydroxymethyl cellulose, polyacrylic acid, polyvinyl alcohol, polyethylene glycol (PEG), polyethylene oxide (PEO), ethylene oxide-propylene oxide co-polymer, polyvinylpyrrolidone (PVP) and/or any other appropriate material.

In general, it may be desirable for the coating to be resilient and adapted to preserve any substance or agent provided by the coating.

In an exemplary embodiment, the water soluble carrier included is pullulan. Pullulan is a soluble polysaccharide polymer derived from a fungal organism composed of triose sugar units. Pullulan coatings may be a self adherent, flexible and/or relatively high strength film that may resiliently remain on the applied surface and that dissolves very quickly on contact with a wet or moist environment. They are also useful in encapsulating other materials, as pullulan has oxygen barrier properties and is thus further useful for preserving an encapsulated material.

In general, a solution of pullulan may be made in water and medicaments and additives may be mixed into the solution to disperse them. The solution may then be applied to the surface of the strip of a dental implement, as discussed before, which upon drying may form an adherent film with encapsulated medicament. The coating may then dissolve on contact with a wet or moist environment such as the oral cavity during use and release the encapsulated medicament and/or other additives.

In some embodiments, it may be desirable for the coating to have a slower rate of dissolution such that the release of medicament and/or other additives may be prolonged. It may further be desirable for only a portion of the coating to dissolve at any one time such that the dental implement may be used more than once with the action of the coating. The coating may, for example, incorporate additives that may serve to slow the rate of dissolution of the water soluble carrier.

In one embodiment, pullulan may be mixed with a relatively insoluble additive to form a coating that may be erodible, or slowly dissolving, if desired. The degree of solubility or erodibility depends on the ratio of pullulan and the insoluble additive. The combination may be, for example, pullulan and high molecular weight PEG or PEO.

The substance or agent carried on the strip may include a variety of useful compositions. For example, the coating may be an antimicrobial composition. Antimicrobial agents may be employed to retard or kill microbes on the strip 10 and the oral cavity by contact and/or deposition of the agent in the oral cavity. These antimicrobial agents may aid in eliminating, preventing or diminishing halitosis, or symptoms of bad oral health. Antimicrobial agents may include, but are not limited to, antibiotics such as β-lactams (e.g. penicillin), aminoglycosides (e.g. streptomycin) and tetracylcines (e.g. doxycycline), antimycotics such as polyene drugs (e.g. amphotericin B) and imidazole and triazole drugs (e.g. fluconazole), and general antimicrobial agents such as quaternary ammonium cations (e.g. benzalkonium chloride or cetylpyridinium chloride (CPC)) and compounds such as triclosan and chlorhexidine.

Other materials in the coating may include flavorants, including sweeteners such as various sugar alcohols (e.g. xylitol), artificial sweeteners (e.g. aspartame) and artificial flavors (e.g. vanillin).

In other embodiments, the substance included on the strip 10 and/or in the coating may be a chelating agent. Chelating agents may aid in promoting microbial detachment by chelating metal ions required for certain microbial binding interactions, such as, for example, calcium ion-dependent cadhedrin attachment, magnesium ion-dependent cross-linking of microbial extracellular matrix (ECM) and/or any other metal ion-dependent microbial attachment mechanisms. Chelating agents included on the tongue scraper may include, but are not limited to, EDTA, ascorbate, porphorin derivatives, and/or any other appropriate chelating agents. It is generally desirable for such agents to be non-toxic and well-tolerated by humans.

In some embodiments, general breath fresheners may be included. Breath fresheners may include, for example, essential oils. Breath freshening compositions, such as those found in mouthwashes, may also be utilized. Examples include the compositions found in products marketed under the trade name BreathRx from Discus Dental, Inc.

In further embodiments, antimicrobial activity may be achieved by utilizing the antimicrobial properties of various metals, especially transition metals which have little to no effect on humans. Examples may include sources of free silver ions, which are noted for their antimicrobial effects and few, if any, biological effects on humans. Metal ion sources may be included in the coating of the strip. The coating may then release free metal ions during use that may produce an antimicrobial effect.

In still further embodiments, the coating may include a de-sensitizing agent, for example. The desensitizing agents may include alkali metal salt including a potassium salt, a sodium salt, a lithium salt or mixtures thereof, including alkali nitrates such as potassium nitrate, sodium nitrate and lithium nitrate, and other potassium salts such as potassium chloride, potassium citrate and potassium bicarbonate; or alkaline metal salts including calcium salts and strontium salts. The salt is, for example, water soluble or at least sparingly water soluble, and the appropriate anions may be chosen to facilitate in their solubility. For example, the appropriate anions may include chlorides, carbonates, nitrates, saccharins, phosphates or mixtures thereof. For example, the alkali metal salt may include at least one potassium salt, or sodium salt, and more for example, potassium nitrate, sodium nitrate, potassium chloride, potassium bicarbonate or sodium saccharin.

In yet still further embodiments, the coating may include remineralizing agents, which may be especially useful with dental implements working on the teeth. Such remineralizing agents may include, for example, sources of calcium, fluoride and/or any other appropriate remineralizing agents.

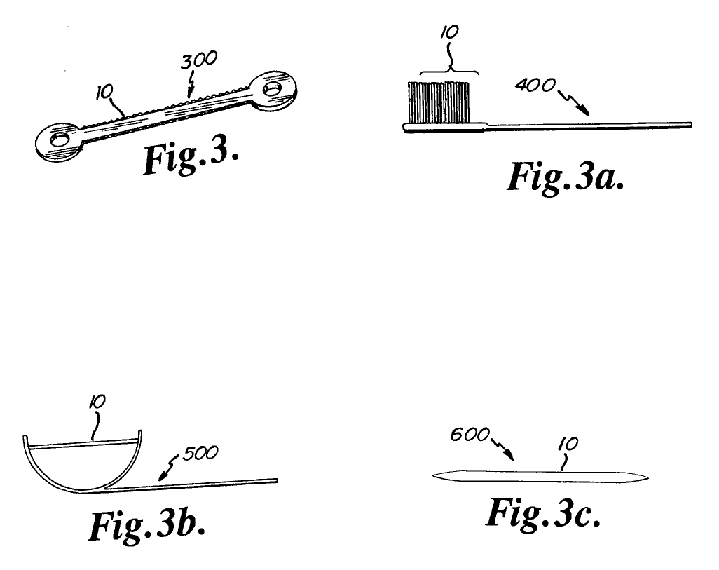

The strip 10 may be adapted to form at least a part of a dental implement. Dental implements may include, but are not limited to tongue scrapers, such as the scraper 300 illustrated in FIG. 3, the bristles of a toothbrush 400, as illustrated in FIG. 3a, the filament of a dental floss 500, as illustrated in FIG. 3b, or as a toothpick 600, as illustrated in FIG. 3c. In general, the strip portions 10 of the illustrated dental implements may be constructed of an appropriate material and possess a coating such as the coatings described above. The coatings may retain and deliver desired substances or agents to the oral cavity as the coating is exposed to the moist environment therein. In some embodiments, the coating may be localized to a specific portion of the strip 10 that may be the working portions of the strip 10.

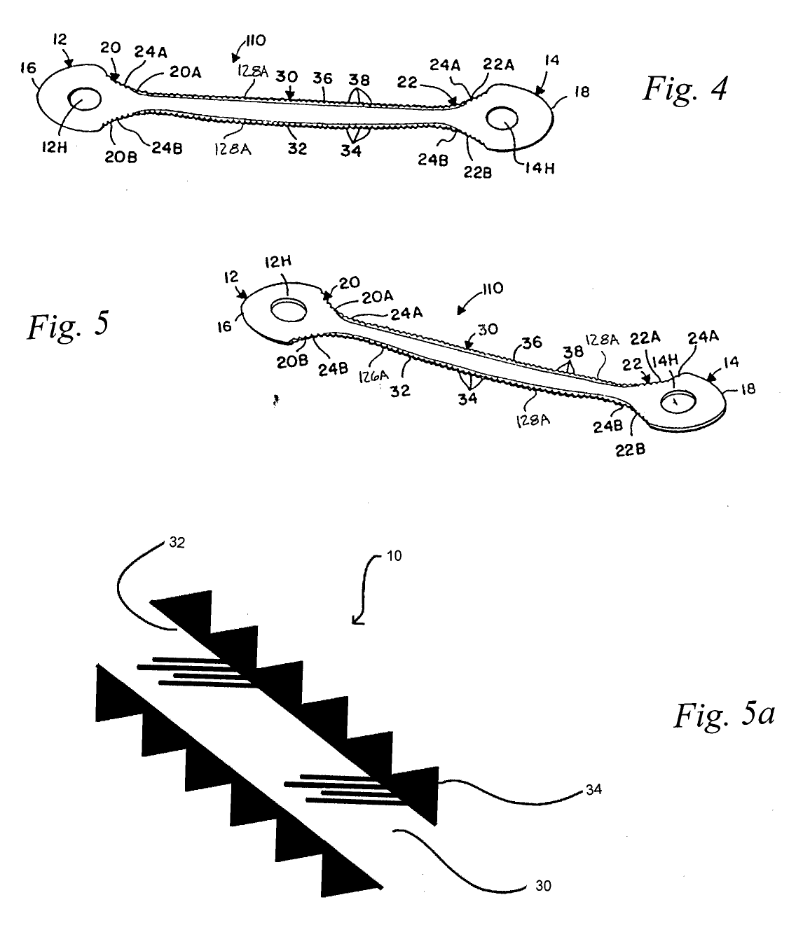

Referring to FIGS. 4 and 5, there is shown a serrated edge dual action tongue scraper 110 adapted for scraping coating material such as food debris, volatile sulfur compounds, dead cells and microflora from the upper surface of a tongue. The tongue scraper 110 includes opposed first and second generally planar end portions 12 and 14, respectively, each terminating distally in an edge 16 and 18, respectively, and each having therethrough a hole 12H and 14H, respectively. Each end portion 12 and 14 extends proximally in a neck 20 and 22, respectively, with each neck having first and second arcuate edges 20A, 20B, 22A and 22B, respectively, tapering to a predetermined width. The edges 20A, 22A, 20B and 22B have a plurality of serrations 24A and 24B, respectively. The tongue scraper 110 further includes a generally planar central portion 30 disposed symmetrically between and smoothly contiguous to the end portion necks 20 and 22. The central portion 30 is defined longitudinally by a convexly arcuate first edge 32 having a multiplicity of serrations 34 formed thereon, and a generally linear second edge 36 having a multiplicity of serrations 38 thereon.

In general, the tongue scraper 110 may be composed of generally disposable material. In one aspect, the material is a polymeric material. In exemplary embodiments, the tongue scraper may be composed of biopolymer material. Biopolymers may be derived from an organism or may be produced synthetically. In general, biopolymers may be advantageous due to their biodegradation and biocompatibility properties. Suitable biopolymers may include those composed of biocompatible monomers, such as glucose, lactic acid, glycolic acid and/or other appropriate monomers. It may be advantageous to utilize such materials as their degradation generally may not produce any toxic or otherwise undesirable products. Exemplary materials for the tongue scraper may include, but is not limited to, cellulose, polylactic acid (PLA), polyglycolic acid (PGA), and/or other biopolymeric material that may be appropriate. In general, it may be desired to produce the tongue scraper from a material that may be economically advantageous, degradable and/or recyclable, particularly biodegradable, and biocompatible.

In one exemplary embodiment, the primary material for the tongue scraper 110 may be a wood or plant fiber material such as, for example, paper, card stock, cardboard and/or any other appropriate wood or plant fiber material. The material may be present as a single layer in the tongue scraper, or it may be present as multiple layers or laminated. The fiber material may be processed in a manner such that the product contains little or no toxic and/or otherwise undesirable additives. Such fiber materials may also be generally advantageous as they are commonplace, with a broad number of products and industries that utilize them for household and personal use, including tissue paper and paper towels. Fiber materials are also generally recyclable, biodegradable and in some senses renewable as they may be derived from serially cultivated and harvested plant species.

In another embodiment, the tongue scraper 110 may be composed of a polypeptide or protein material. Such materials are typically biocompatible and biodegradable as they are derived from organisms, including, but not limited to, mammals. Such materials include, but are not limited to, silk, collagen and/or other polypeptide or protein materials.

In general, desirable materials may be fibrous and/or otherwise exhibit desirable properties such as high tensile strength, insolubility in water and biological fluids, such as saliva, flexibility and elasticity.

In a further embodiment, the material of the tongue scraper 110 may be assembled in an intertwining fashion, such as being woven, spun, coiled, and/or otherwise formed into a matrix-like configuration. Such configuration may be utilized to increase the mechanical stability of the tongue scraper and may alter its absorptivity.

In an exemplary embodiment, the tongue scraper material may be coated, impregnated or otherwise include a substance or agent that may serve in part to bind the structure of the tongue scraper together prior to use. The substance or agent may be present in the entirety of the tongue scraper or it may be present in only certain portions such as, for example, the edges. The substance or agent may be adapted to at least partially dissolve and/or dissipate from the tongue scraper structure. The tongue scraper may also be adapted to break down and/or at least partially degrade after or as the substance or agent has dissolved or dissipated from the tongue scraper structure. The substance or agent may include any desirable materials, which will be discussed in further detail below.

FIG. 5a illustrates an embodiment of a tongue scraper 110 that includes erodible and/or dissolvable portions. The generally planar portion 30 may include edge 32 which may have formed on it serrations 34. The edge 32 and serrations 34 may be formed from a different material than the generally planar portion 30 of the tongue scraper 10. The material may be generally erodible and/or dissolvable such that during the course of use, the edge 32 and serrations 34 may dissipate and/or degrade from the tongue scraper 10. The material may also include a medicament.

The material may include a film forming and/or soluble material for coating, impregnating and/or otherwise including with the tongue scraper. Suitable materials may include hydrophilic materials including polysaccharides such as carrageenan, chondroitin sulfate, glucosamine, pullulan, soluble cellulose derivatives such as hydroxypropyl cellulose and hydroxymethyl cellulose, polyacrylic acid, polyvinyl alcohol, polyethylene glycol (PEG), polyethylene oxide (PEO), ethylene oxide-propylene oxide co-polymer, polyvinylpyrrolidone (PVP), polycaprolactone, polyorthoesters, polyphosphazene, polyvinyl acetate, and polyisobutylene.

The material of the tongue scraper or portions thereof may be adapted to have a desirable rate of erosion and/or dissolution. The rate may be modified by the inclusion of hydrophobic and/or less soluble additives. Suitable materials may include, but are not limited to, those from the family of quaternary ammonium acrylate/methacrylate co-polymers, (Eudragit RS), cellulose and its lower solubility derivatives, such as butyl cellulose, hydroxybutyl cellulose, and ethylhydroxyethyl cellulose, high molecular weight PEG or PEO, or a combination thereof.

Similar materials and/or combinations may further be utilized to imbue the tongue scraper base material with erodible and/or dissolvable characteristics such that the tongue scraper may degrade during the course of its use. In embodiments where medicament may be included in the base material of the tongue scraper, such characteristics may further allow the medicament to be released in a controlled fashion.

In yet still other embodiments, the tongue scraper material may incorporate substances that may promote its degradation or deformation after use. For example, water soluble polymers may be incorporated into the tongue scraper material as a disintegrant such that after a period of time, the tongue scraper material loses shape and/or integrity after prolonged contact with a moist environment such as the oral cavity. The material of the tongue scraper may also be adapted such that only soluble, innocuous materials are left in the oral cavity during the course of degradation or deformation of the tongue scraper.

In another embodiment, a medicament may be present as a layer or multiple layers within a laminated structure of a tongue scraper.

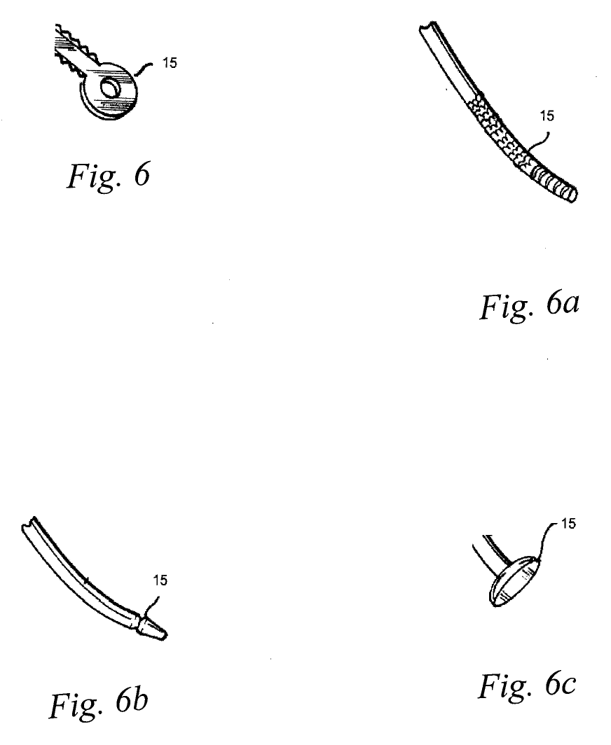

In some aspects, the tongue scraper 110 may feature formations that may be adapted to increase the ease and efficiency of its handling by the user. FIGS. 6, 6a, 6b and 3c illustrate various embodiments of handling features 15 that may form the end portions 12, 14 of the tongue scraper 10 of FIGS. 4 and 5. The end portions 12, 14 may include, for example, ring handles, such as those shown in FIGS. 4, 5 and 6, textured handles, such as shown in FIG. 6a, or handle bars, either collinear or offset from the axis of the tongue scraper 10, such as shown in FIGS. 6b and 6c, respectively.

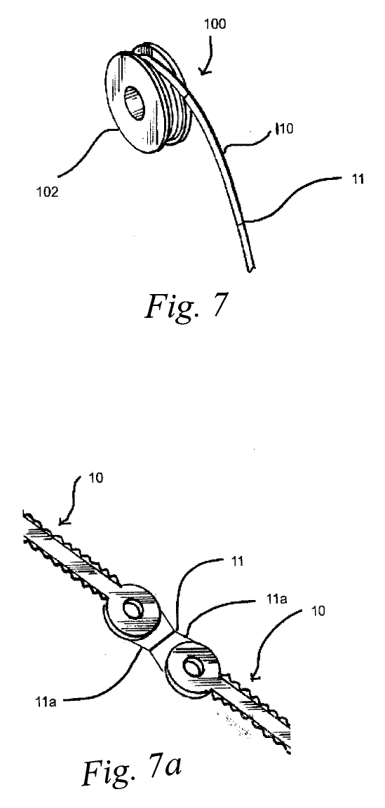

In one exemplary aspect, tongue scrapers 110 may be provided in a continuous length. FIG. 7 illustrates a roll of tongue scrapers 100 in one embodiment of the invention. The individual tongue scrapers 110 may be part of a continuous length that may be separated by dividing region 11 between each tongue scraper 110. The roll of tongue scrapers 100 may further include a spool 102 around which the tongue scrapers 110 may be wrapped for compact storage and dispensing.

For use, an individual tongue scraper 110 may be unrolled from the spool 102 and torn off or otherwise removed from the continuous length at dividing region 11. The dividing region 11 may include any feature that may ease the removal of a tongue scraper 110 from the roll and may include, but is not limited to, perforations, a region of reduced thickness or width, a pre-stressed region and/or any other appropriate feature. In some embodiments, a leader section 11a may be included on either side of the dividing region 11. The leader sections 11a may feature increased flexibility such that they may facilitate the spooling of the length of tongue scrapers 110. The leader sections 11a may further serve as a buffer against damage to the individual tongue scrapers 110 when torn off for use.

In some embodiments, the tongue scrapers 110 may be spooled with an intervening layer or material. A backing layer or material may be included such that the planar surfaces of the tongue scrapers 110 do not touch when spooled. This may decrease the chance of any contamination or other spoiling of the tongue scrapers 110 on the spool 102 prior to their use.

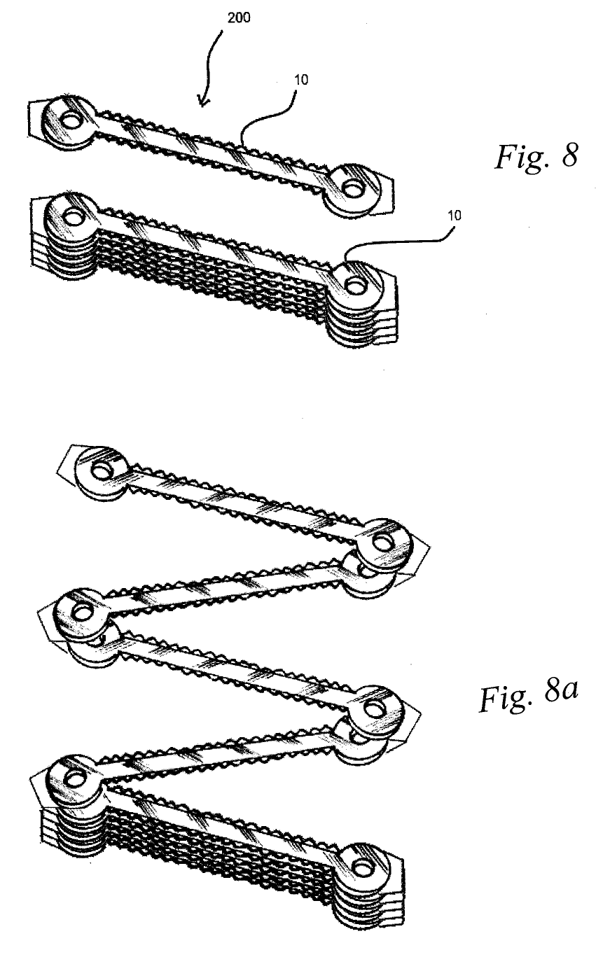

In another aspect, the tongue scrapers 110 may be provided in a stack. FIG. 8 illustrates a stack of individual tongue scrapers 110 in one embodiment of the invention. The stack 200 may include a plurality of tongue scrapers 110, which may be stacked with their planar sections together such that the stack may occupy the smallest volume during storage.

In another embodiment, the individual tongue scrapers 110 may be part of a continuous length in a manner similar to that shown in FIGS. 7 and 7a. The individual tongue scrapers 110 may then be, instead of spooled, stacked by folding the dividing regions 11 in opposing directions at every section, forming a zig-zag stack 300, as illustrated in FIG. 8a.

In still another aspect, the tongue scraper 110 may be adapted to carry substances for action on and/or delivery to the oral cavity.

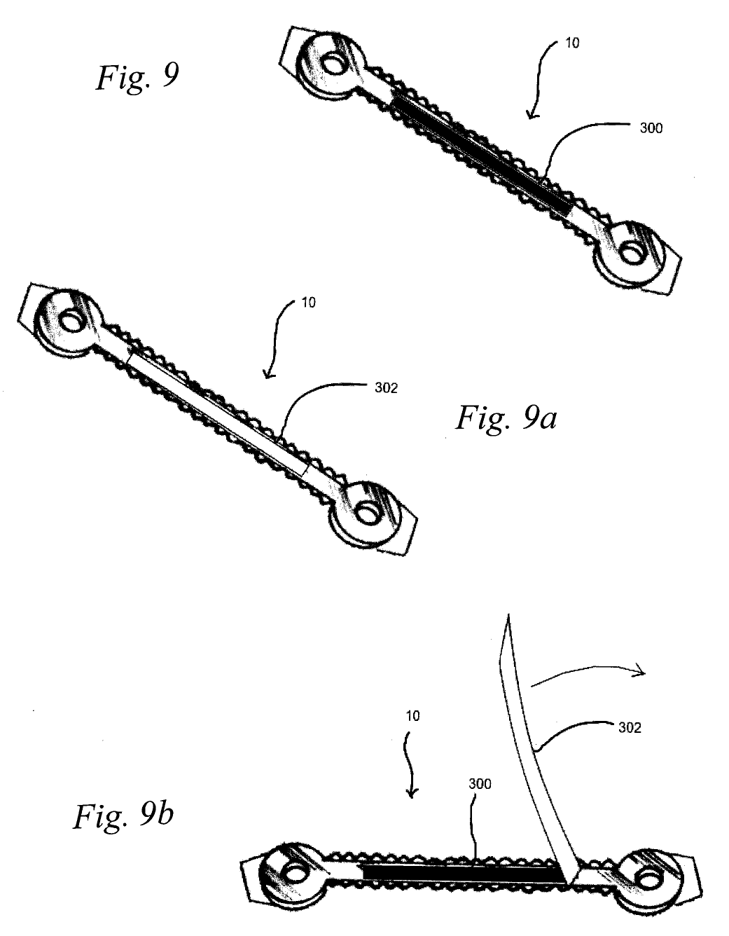

In some embodiments, the tongue scraper 110 may include various coatings and/or treatments that may facilitate its cleaning functions, application of a medicament, and/or help maintain sterility during use and storage. FIG. 9 illustrates an embodiment where at least a portion of the surface of the tongue scraper 110 is coated with a medicament 300. The medicament 300 may be present on any desirable portion of the tongue scraper 110 and may in general be present on portions that may interact with the oral cavity, particularly the tongue. The medicament 300 may also be present on the entire tongue scraper 110.

In further embodiments, the medicament 300 may be covered and/or otherwise isolated prior to use. FIGS. 9a and 9b illustrate an embodiment where the medicament 300 is covered by a removable strip 302 prior to use. The strip 302 may then be removed to expose the medicament 300. In general, a variety of isolating features may be employed, which include, but are not limited to, removable strips and films, degradable barriers, dissolvable barriers, sheaths and/or any other appropriate isolating feature or combinations thereof.

The medicament present on the tongue scraper 110 may include a variety of useful compositions. For example, the tongue scraper 110 may be coated with an antimicrobial composition. Antimicrobial agents may be employed to retard or kill microbes on the tongue scraper 110 and the tongue by contact and/or deposition of the agent on the tongue. These antimicrobial agents may aid in eliminating, preventing or diminishing halitosis, or symptoms of bad oral health, as noted above. Antimicrobial agents may include, but are not limited to, those listed above including β-lactams (e.g. penicillin), aminoglycosides (e.g. streptomycin) and tetracylcines (e.g. doxycycline), antimycotics such as polyene drugs (e.g. amphotericin B) and imidazole and triazole drugs (e.g. fluconazole), and general antimicrobial agents such as quaternary ammonium cations (e.g. benzalkonium chloride or cetylpyridinium chloride (CPC)) and compounds such as triclosan and chlorhexidine.

The composition may include a binding agent or carrier, an antimicrobial agent and/or other materials conducive to its retention on the tongue scraper and its use as a sterilizing agent.

The medicament may include a film forming and/or soluble material for coating, impregnating and/or otherwise including the medicament with the tongue scraper. Suitable materials may include those listed before, including hydrophilic materials including polysaccharides such as carrageenan, chondroitin sulfate, glucosamine, pullulan, soluble cellulose derivatives such as hydroxypropyl cellulose and hydroxymethyl cellulose, polyacrylic acid, polyvinyl alcohol, polyethylene glycol (PEG), polyethylene oxide (PEO), ethylene oxide-propylene oxide co-polymer, polyvinylpyrrolidone (PVP), polycaprolactone, polyorthoesters, polyphosphazene, polyvinyl acetate, and polyisobutylene.

The medicament may be adapted to have a desirable rate of erosion and/or dissolution. The rate may be modified by the inclusion of hydrophobic and/or less soluble additives in varying proportion. Suitable materials may include, but are not limited to, those from the family of quaternary ammonium acrylate/methacrylate co-polymers, (Eudragit RS), cellulose and its lower solubility derivatives, such as butyl cellulose, hydroxybutyl cellulose and ethylhydroxyethyl cellulose, high molecular weight PEG or PEO or a combination thereof. Slow release materials may also be utilized such that a substance or agent within the medicament may simply leach out while the carrier largely retains its integrity. Such materials include, but are not limited to, PLA, PGA, copolymers thereof, polycaprolactone, and/or any other suitable material.

In some embodiments, the medicament may be adapted to dissolve on contact with the moist tongue and oral cavity, as described above, thus allowing dispersal of a substance or agent quickly. Water soluble carriers such as, for example, hydroxypropylcellulose, polyvinylpyrrolidone (PVP), pullulan or carrageenan may be employed to effect such action.

Other materials in the coating may include flavorants, including sweeteners such as various sugar alcohols (e.g. xylitol), artificial sweeteners (e.g. aspartame) and artificial flavors (e.g. vanillin), as noted before.

In other embodiments, the substance included on the tongue scraper may be a chelating agent, as discussed above, to aid in promoting microbial detachment by chelating metal ions required for certain microbial binding interactions, such as, for example, calcium ion-dependent cadhedrin attachment, magnesium ion-dependent cross-linking of microbial extracellular matrix (ECM) and/or any other metal ion-dependent microbial attachment mechanisms. Chelating agents included on the tongue scraper may include, but are not limited to, EDTA, ascorbate, porphorin derivatives, and/or any other appropriate chelating agents. It is generally desirable for such agents to be non-toxic and well-tolerated by humans.

It may further be generally desirable for a carrier to provide sufficient encapsulating and/or preservation properties such that the substances or agents applied in the coating may be retained and preserved prior to use. Pullulan, for example, may be utilized as an oxygen barrier to aid in preserving any substances or agents in the coating.

In other embodiments, to increase the coating efficiency, the surfaces to be coated may be roughened or pitted. The surfaces may also be chemically modified such that they promote attachment and/or retention of an applied substance.

In further embodiments, antimicrobial activity may be built into the tongue scraper itself by, for example, covalently bonding antimicrobial agents to the surface of the tongue scraper. These covalently bonded materials may act to minimize microbial growth on the tongue scraper 10. In addition, any microbial organisms that may chance to be attached to the material may be killed by interaction with the coating. For example, quaternary ammonium cations, such as N-alkyl-pyridiniums, may be used as antimicrobial moieties in covalently attached polymeric surface coatings. In one case, poly(4-vinyl-N-hexylpyridinium) (N-alkylated-PVP) was previously noted to have an optimum alkyl side chain length for antimicrobial activity. The side chain length of the alkyl group may, for example, vary from 0 (to side chain) to 12 carbons long, more for example from 5 to 7 carbons long. The alkyl side chains may provide increased hydrophobicity for the coating and may promote association with microbial membranes. Polyethylenimine (PEI) was also previously used as a bacteriocidal coating when both N-alkylated on its primary amino group and subsequently N-methylated on its secondary and tertiary amino groups to raise the overall number of cationic quaternary amino groups. An increased number of cationic groups (permanently charged or charged due to the pH of the system) may promote an electrophoretic mechanism when associated with microbial membranes, which may promote the lysis of the microbe. Any such covalently bonded quaternary ammonium cation polymeric coatings may be used to give an antimicrobial property to the tongue scraper surface.

Antimicrobial coatings may be covalently attached to the surface by a variety of methods and may include, for example, creating suitable reaction sites, such as free hydroxyl or amino groups, by coronal discharge, surface etching, hydrolyzation or other methods that disrupt the surface of the tongue scraper to create sites of suitable reactivity. The antimicrobial coatings may then be synthesized by reacting the various precursors with the prepared surface of the tongue scraper to build the proper coating. In other cases, silanes may be used as coupling agents to complex antimicrobial moieties to the tongue scraper.

In yet further embodiments, antimicrobial activity may be achieved by utilizing the antimicrobial properties of various metals, especially transition metals which have little to no effect on humans. Examples may include sources of free silver ions, which are noted for their antimicrobial effects and few, if any, biological effects on humans. Metal ion antimicrobial activity may be created by a variety of methods that may include, for example, mixing a source of a metal ion with the polymer of the tongue scraper during manufacture, coating the surface by methods such as plasma deposition, loosely complexing the metal ion source by disrupting the surface of the tongue scraper to form affinity or binding sites by methods such as etching or coronal discharge, and depositing a metal onto the surface by means such as electroplating, photoreduction and precipitation. The tongue scraper may then slowly release free metal ions during use that may produce an antimicrobial effect.

In still further embodiments, the tongue scraper may be coated with a de-sensitizing agent, for example. The desensitizing agents may include alkali metal salt including a potassium salt, a sodium salt, a lithium salt or mixtures thereof, including alkali nitrates such as potassium nitrate, sodium nitrate and lithium nitrate, and other potassium salts such as potassium chloride, potassium citrate and potassium bicarbonate; or alkaline metal salts including calcium salts and strontium salts. The salt is, for example, water soluble or at least sparingly water soluble, and the appropriate anions may be chosen to facilitate in their solubility. For example, the appropriate anions may include chlorides, carbonates, nitrates, saccharins, phosphates or mixtures thereof. For example, the alkali metal salt may include at least one potassium salt, or sodium salt, and more for example, potassium nitrate, sodium nitrate, potassium chloride, potassium bicarbonate or sodium saccharin.

In some embodiments, general breath fresheners may be included with the tongue scraper. Breath fresheners may include, for example, essential oils. Breath freshening compositions, such as those found in mouthwashes, may also be utilized. Examples include the compositions found in products marketed under the trade name BreathRx from Discus Dental, Inc.

In other embodiments, the tongue scraper 10 may include both substances that are coated onto the tongue scraper 10 as a means of dispensing them onto the tongue and pre-applied ones for helping to reduce and/or prevent microbial growth or other contamination on the tongue scraper 10.

The coating may be present over substantially the entire exposed surfaces of the tongue scraper 10, or it may be present only on the surfaces that come into contact with the tongue. In the embodiments where the coating is only present on the surfaces that come into contact with the tongue, the surfaces may be roughened to hide the material inside the valleys or pits, as noted above.

In some aspects, the tongue scrapers may be stored and dispensed in individual packages. Packaging may include, but is not limited to, paper wrappers, plastic wrappers, foil wrappers, and/or any other appropriate packaging. It may be generally desirable for the packaging to be sealed such that the contents may be well isolated from the environment prior to use. It may also be generally desirable to utilize packaging that is not bulky and may occupy a very small volume during storage. The packaging may also incorporate features such that it may be easily opened by a user while still retaining its sealing properties during storage.

In other aspects the tongue scrapers may be stored and dispensed from a dispensing device. A dispensing device may, in general, store the tongue scrapers and dispense them in a manner that enables the user to easily isolate a single scraper for use.

The dispensing device may include an opening at one end or along one side. When the opening is at the end, the tongue scraper may be pulled out as an extension of the dispensing device, or perpendicular to the dispensing device. After use, it may be torn off and disposed of to reduce cross-contamination. When the opening is along one side, the tongue scraper may be pulled out in a perpendicular direction. The opening may further be fitted with a door. The roll may be fixedly or slidably attached to an end of the dispensing device or anywhere inside the housing.

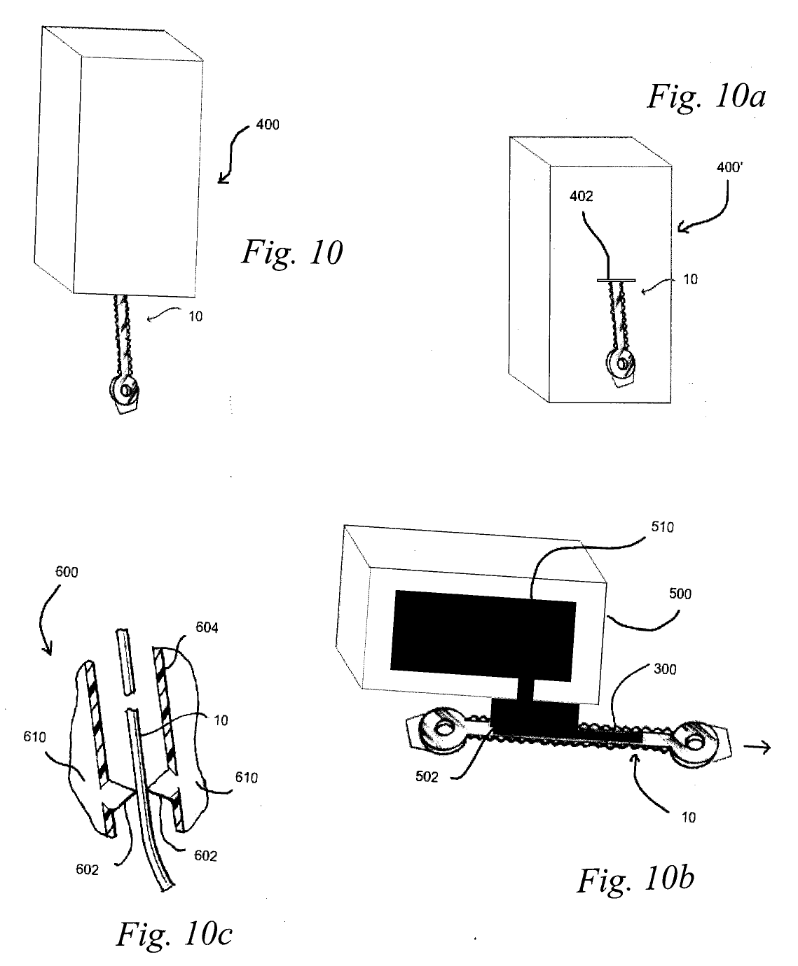

In one embodiment, as illustrated in FIG. 10, a dispensing device 400 may be adapted to store and dispense tongue scrapers 110. The tongue scrapers 110 may be present in a roll, such as shown in FIG. 4, or they may be present as a stack as in FIG. 8. The dispensing device 400 may incorporate a variety of features that may aid in storing and dispensing the tongue scrapers 110, such as, for example, a mounting pin for retaining a roll of tongue scrapers or a spring-loaded platform such that a stack of tongue scrapers will be accessible via an aperture. The dispensing device 400 may also be adapted to store and dispense tongue scrapers that are individually packaged.

In another embodiment, as illustrated in FIG. 10a, a dispensing device 400′ may be adapted to store and dispense tongue scrapers 10 that may be present in a zig-zag stack, as illustrated in FIG. 8a. An aperture 402 may be present on the surface of the dispensing device 400′ such that it lies at the approximate middle of the stack of tongue scrapers 110. This configuration may be utilized to effectively dispense tongue scrapers 110 in a zig-zag stack such that when a tongue scraper 110 is pulled out, the next tongue scraper 110 may be pulled into position.

In a further aspect, as also mentioned above, the housing may include features that may coat the tongue scraper as it is being pulled out of the housing with any substance as discussed.

In some aspects, a device may be adapted provide tongue scrapers with a substance on their surface(s) just prior to use. In one embodiment, as illustrated in FIG. 7b, a medicament 300 may be applied to a tongue scraper 110 by an application device 500. The application device 500 may be free-standing, or, in an exemplary embodiment, it may be included as part of a dispensing device. The application device 500 may include, for example, a substance applicator 502 which may be connected to a reservoir 510 which may be adapted to store the medicament 300.

In some embodiments, the medicament 300 may be a liquid with relatively low viscosity. A substance applicator 502 may be adapted to apply a layer of the medicament 300 onto the surface of a tongue scraper 110. The applicator 502 may be any appropriate device, material or object that may effectively retain a substance from reservoir 510 and deliver it to the surface of a tongue scraper 110. The applicator 502 may be, for example, a sponge such that it may absorb medicament 300 from the reservoir 510, effectively deliver it to the tongue scraper 110 while still retaining the medicament 300 when not in contact with a tongue scraper 110.

In other embodiments, the medicament 300 may be relatively viscous and/or gel-like. An applicator 502 may be adapted to effectively deliver the medicament 300 to the tongue scraper 110. In one embodiment, a gel-like medicament 300 may be retained within the aperture of an applicator 502 by its own viscosity or surface tension. The medicament 300 may then be delivered to the tongue scraper 110 by contact actions, such as, for example, shear force or absorbance by the tongue scraper 110.

The medicament 300 may also be of a relatively solid consistency. In general, it may be desirable for the medicament 300 to be soft and/or otherwise amenable to coating a tongue scraper 110. In one embodiment, the medicament 300 may be a solid that may be contacted by the surface of the tongue scraper 110 such that a desired amount of the medicament 300 may be transferred to the tongue scraper 110. The tongue scraper 110 may be textured, roughened and/or otherwise adapted to retain at least a portion of the medicament 300 present on the applicator 502.

In some embodiments, the tongue scraper may be provided with a medicament on more than one side. FIG. 10c illustrates an embodiment of an application device 600 that may be adapted to provide a medicament to at least one surface or edge of the tongue scraper 110. The application device may operate in a manner similar to the embodiments of application devices discussed above. The application device 600 may include at least one applicator 602, each of which may contact at least a portion of the tongue scraper 110. The applicators 602 may be connected to at least one reservoir 610 that may be adapted to store and deliver a medicament to the applicators 602. The tongue scraper 110 may be present in a hollow portion 604 of the application device 600. The hollow portion 604 may be adapted to mount the applicators 602 as well as position them in such a way that they contact the appropriate portions of the tongue scraper 110 during operation.

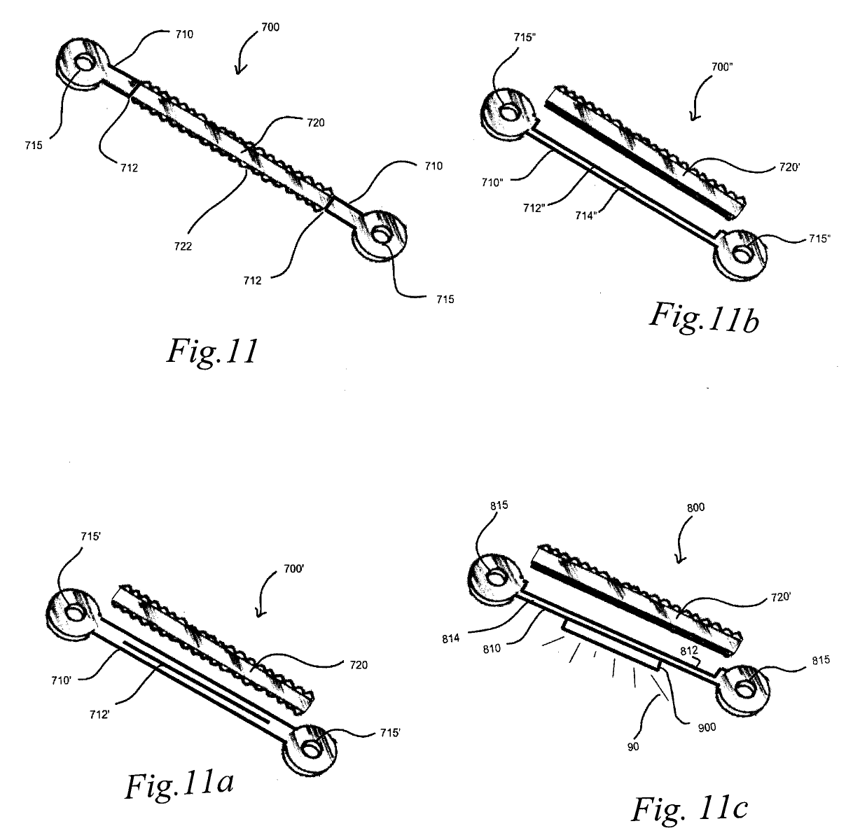

In some aspects, a tongue scraper may be present as a compound device. FIG. 11 illustrates a tongue scraper device 700. The tongue scraper device 700 may include a set of handles 710 that may be adapted to releasably retain a tongue scraping insert 720. The insert 720 may, for example, be a disposable tongue scraper that may be substantially identical or similar to the tongue scrapers discussed above. The tongue scraping insert 720 may lack independent handling features and may be releasably retained by the set of handles 710 for articulation and use. The handles 710 may be incorporate handling features 715 which may include, but are not limited to, ring handles, handle bars and textured grips, similar to those shown in FIGS. 6, 6a, 6b and 6c. The handles 710 may also include retaining features 712 which may be adapted to releasably retain the insert 720. The handles 710 may be adapted to be reusable and may be coupled to disposable inserts 720. This may aid in reducing overall waste generated while still retaining the desirable traits of a disposable tongue scraper.

In another embodiment, a tongue scraping device may be adapted to support a tongue scraping insert. FIG. 11a illustrates a tongue scraping device 700′ with handle 710′ and tongue scraping insert 720. The handle 710′ may be substantially similar or identical to the tongue scrapers discussed above, but the handle 710′ may be adapted to lack specific tongue scraping features. The handle 710′ may rather be adapted to releasably retain an insert 720 via at least one interface feature 712′ in manners similar to those discussed above. The handle 710′ may further be adapted to support the insert 720. The handle 710′ may be constructed of a material that may be more durable, flexible and/or otherwise amenable to being reused and for serving as a support structure for an insert 720.

In some embodiments, the handle of the tongue scraping device may be substantially the same or similar in width to the insert. In other embodiments, the handle may feature a different width than the insert.

FIG. 11b illustrates an embodiment of a tongue scraping device 700″ that may feature a reduced width member 714″. The reduced width of the member 714″ may aid in decreasing the overall weight and/or perceived bulkiness of the handle 710″ while still serving to reinforce the insert 720′ during use. The handle 710″ may also include an interface feature 712″ that may be adapted to releasably retain the insert 720′ during use. The interface feature 712″ may be adapted to mount either a dual working edge insert, such as the insert 720 of FIGS. 11 and 11a, or a single working edge insert 720′.

In some embodiments, the tongue scraping insert may be adapted to be adhesively retained by the handle of the tongue scraping device. The tongue scraping inserts may, for example, include pressure sensitive adhesive materials in its makeup. Pressure sensitive adhesive materials may include, but are not limited to, acrylic and methacrylate adhesives, rubber-based pressure sensitive adhesives, styrene copolymers (SIS/SBS), silicones, and gum or other polysaccharide materials, such as pullulan and carrageenan.

Any of the tongue scraping inserts described above may include any of the various coatings, impregnations or inclusions discussed above or they may be constructed to include any of the discussed substances or agents in a manner similar to individual tongue scrapers.

In some embodiments, the handle of a tongue scraping device may incorporate features that may aid in reducing the physical space required for storage. For example, the handle may feature hinges, be assembled from multiple smaller components or otherwise be adapted to take up less space during storage. In an exemplary embodiment, the handle may be constructed of materials that may allow it to be bent and/or deformed extensively while incorporating sufficient memory or reforming properties such that it may be restored to the original shape easily. Some materials that may be utilized include, but are not limited to, high strength flexible thermoplastics, artificial and synthetic elastomers, titanium and titanium alloys, and/or any other material that may exhibit the desired memory, flexibility and/or reforming properties.

Any of the above described devices may also be fitted with a light source. In some aspects, a light source may be included to act on the oral cavity directly or to enhance the therapeutic effects of the tongue scraper or other dental implements, its coatings or included medicaments. The light source may also have therapeutic effects or may be adapted to activate and/or otherwise affect the medicament applied to the tongue scraper. A light source may, for example, act on a photointiator present in the composition of the applied medicament.

FIG. 11c illustrates an embodiment of a tongue scraping device 800 that may include a light source 900. The light source 900 may be adapted to emit electromagnetic radiation 90 of any desired wavelength or combination of wavelengths such that they may enhance any therapeutic effects described above. The electromagnetic radiation 90 may also be utilized to act directly upon microbes by, for example, killing, disrupting cellular processes or disrupting cellular structures.

The light source 900 may be integral to the tongue scraping device 800 or it may be separate and/or attachable. In one embodiment, a light source may be included by constructing the handle 810 at least partially from a luminescent substance or it may include substances that may produce luminescence when combined or otherwise triggered. The handle 810 may, for example, include an internal channel or chamber that may house at least one chemical that may be luminescent. Multiple chemicals may also be contained and/or segregated within the handle 810 and combined to produce luminescence during use. Chemicals may be contained within breakable tubes, similar to those present in disposable chemical glow sticks, such that when the handle 810 is bent sufficiently, the tubes may break and combine the chemicals within the handle 810 to produce luminescence.

The descriptions above in relationship to a tongue scraper are only exemplary and are also applicable to other dental implements.

Having described the present invention in reference to the embodiments mentioned above, it will be understood that any variations and equivalents are within the scope of the present invention.

1. A dental implement comprising:
a strip having a longitudinal edge and a transverse edge, said longitudinal edge is longer than the traverse edge; and
a coating coated on at least one edge of the strip, said coating comprising a mixture of at least one medicament and a water soluble or erodible carrier;
wherein said dental implement comprises a tongue scraper, a toothpick, a dental floss, bristles of a toothbrush or combinations thereof.

2. The dental implement of claim 1 wherein said coating comprises a hydrophilic material and a dissolution modifier.

3. The dental implement of claim 2 wherein said hydrophilic material comprises polysaccharides, soluble cellulose derivatives, polyacrylic acid, polyvinyl alcohol, polyethylene glycol (PEG), polyethylene oxide (PEO), ethylene oxide-propylene oxide co-polymer, polyvinylpyrrolidone (PVP) or mixtures thereof.

4. The dental implement of claim 2 wherein said hydrophilic material comprises pullulan.

5. The dental implement of claim 2 wherein said dissolution modifier comprises at least one hydrophobic material.

6. The dental implement of claim 1 wherein a surface of the edge of the strip is modified for better retain the coating.

7. The dental implement of claim 1 wherein said coating is in a dry or substantially dry state.

8. The dental implement of claim 4 wherein said coating further comprises quaternary ammonium acrylate/methacrylate co-polymers, butyl cellulose, hydroxybutyl cellulose, ethylhydroxyethyl cellulose, high molecular weight PEG or PEO, or a combination thereof.

9. A dental implement comprising:
a strip having a longitudinal edge and a transverse edge, said longitudinal edge is longer than the traverse edge; and
at least one edge of the strip comprises a mixture of at least one medicament and a water soluble or erodible carrier;
wherein said dental implement comprises a tongue scraper, a toothpick, a dental floss, bristles of a toothbrush or combinations thereof.

10. The dental implement of claim 9 wherein said at least one edge comprises pullulan.

11. The dental implement of claim 9 wherein said at least one edge comprises a hydrophilic carrier and a dissolution modifier.

12. The dental implement of claim 11 wherein said dissolution modifier comprises quaternary ammonium acrylate/methacrylate co-polymers, butyl cellulose, hydroxybutyl cellulose, ethylhydroxyethyl cellulose, high molecular weight PEG or PEO, or a combination thereof.

13. The dental implement of claim 9 wherein said implement is individually multiply packaged.

14. A disposable tongue scraper comprising:
a generally planar strip having at least one edge, said edge having sufficient integrity for at least one use; and
present on said at least one edge is a coating comprising a substance that is releasable upon contact with water and/or saliva.

15. The disposable tongue scraper of claim 14 wherein said edge is reinforced by said coating for sufficient integrity to last at least one use.

16. The disposable tongue scraper of claim 14 wherein said substance comprises antimicrobial compositions, flavorings, chelating agents, sweeteners, fluoride agents, breath fresheners, desensitizing agents, or combinations thereof.

17. The disposable tongue scraper of claim 14 wherein said coating comprises a dissolvable coating or erodible coating.

18. The dental implement of claim 14 wherein said coating comprises a hydrophilic material or a mixture of a hydrophilic material and a hydrophobic material.

19. The dental implement of claim 18 wherein said hydrophilic material comprises polysaccharides, soluble cellulose derivatives, polyacrylic acid, polyvinyl alcohol, polyethylene glycol (PEG), polyethylene oxide (PEO), ethylene oxide-propylene oxide co-polymer, polyvinylpyrrolidone (PVP) or mixtures thereof.

20. The dental implement of claim 19 wherein said hydrophilic material comprises pullulan.
